(19) United States
(12) Patent Application Publication
(10) Pub. No.: US 2007/0129584 A1
(43) Pub. Date: Jun. 7, 2007

(54) Metallic compound fixed to a support, method for production and use of said compound in hydrocarbon metathesis reactions

(76) Inventors: Jean-Marie Basset, Caluire (FR); Christophe Coperet, Lyon (FR); Daravong Soulivong, Lyon (FR); Mostafa Taoufik, Rillieux-La-Pape (FR); Jean Thivolle-Cazat, Fontaine-Sur-Saone (FR)

Correspondence Address: STITES & HARBISON PLLC, 1199 NORTH FAIRFAX STREET, SUITE 900, ALEXANDRIA, VA, 22314, US

(21) Appl. No.: 10/550,628

(22) PCT Filed: Mar. 24, 2004
(86) PCT No.: PCT/FR04/00730

§ 371 Date: Jan. 22, 2007

(30) Foreign Application Priority Data
Mar. 25, 2003 (FR) 03/03588

(51) Int. Cl. C07C 2/02; B01J 23/00

(52) U.S. Cl. 585/318; 585/375; 502/305

(57) ABSTRACT

The invention relates to a supported metal compound, comprising a support made from aluminium oxide to which a tungsten hydride is grafted. The support may be selected from the homogeneous supports with a composition based on aluminum oxide and from heterogeneous supports made from aluminium oxide with aluminum oxide essentially on the surface of said support. The support may in particular comprise aluminium oxide, mixed aluminium oxides and modified aluminium oxides particularly comprising one or more elements of groups 15 to 17 of the periodic table of the elements, such as phosphorus, sulphur, fluorine or chlorine. A support made from porous, non-porous or mesoporous aluminas is preferred. The degree of oxidation of the tungsten may have a value of from 2 to 6. The tungsten atom is generally bonded to one or several hydrogen atoms and optionally to one or several hydrocarbon groups. According to the invention, the compound may be prepared by a dispersion step and grafting of a tungsten organometallic precursor to the support made from aluminium oxide then hydrogenation of the resulting product. The product may be used as catalyst in reactions of cleavage and recombination of hydrocarbons, particularly in hydrocarbon metathesis reactions most particularly of alkanes. The product has a surprising, extremely high catalytic activity in this type of reaction and in particular, a high selectivity for the formation of n-alkanes with relation to iso-alkanes.

The present invention relates to a metallic compound fixed on a solid support, to a method for production and to uses of compound in particular as a hydrocarbon compound metathesis reaction catalyst.

International patent application WO 98/02244 describes an alkane metathesis method in which one or more alkanes is/are reacted on a solid compound comprising a metallic hydride grafted onto a solid support. Production of the solid compound comprises firstly grafting an organometallic compound onto a solid support so as to form a grafted organometallic compound, followed by hydrogenolysis treatment of said compound with the assistance of hydrogen or another reducing agent so as to form a metallic hydride grafted onto the support. The metallic hydride produced in this manner is used as a catalyst in alkane metathesis reactions. The metal of the metallic hydride may be selected from among the transition metals of groups 5 to 6 of the periodic table of the elements, and the support may be selected from among numerous solid oxides. The Examples of the international patent application typically describe the production of a tantalum hydride grafted onto silica and the use of this hydride in ethane, propane, butane, or isobutane metathesis reactions. The Examples also describe the production of a tungsten hydride grafted onto silica and the use of this hydride in a propane metathesis reaction. These tantalum or tungsten hydrides grafted onto silica are active in alkane metathesis reactions. However, it has been considered important to find hydrocarbon metathesis reaction catalysts which exhibit even greater activity in this area.

It has surprisingly been found that, among all the possible combinations between, on the one hand, group 5 and 6 transition metals and, on the other hand, supports based on solid oxides, there was nothing to indicate that the specific selection of tungsten as the transition metal and of aluminium oxide as the solid support could give rise to a metallic hydride grafted onto a solid support which was capable of bringing about a considerable improvement in the catalysis of hydrocarbon metathesis reactions. This improvement relates both to a considerable rise in catalytic activity and to an increase in selectivity for the formation of normal hydrocarbons in comparison with “iso” form hydrocarbons in hydrocarbon metathesis reactions.

The present invention first of all relates to a supported metallic compound comprising a support based on aluminium oxide onto which a tungsten hydride is grafted. A tungsten hydride grafted onto a support based on aluminium oxide is generally taken to mean an atom of tungsten attached to at least one hydrogen atom and, in particular by at least one single bond, onto said support.

The periodic table of the elements mentioned above and hereafter is that presented by IUPAC in 1991 in which the groups are numbered from 1 to 18 and which may be found, for example, in “CRC Handbook of Chemistry and Physics”, 76th Edition (1995-1996), by David R. Lide, published by CRC Press, Inc. (USA).

The compound according to the invention essentially comprises a tungsten hydride grafted onto a support based on aluminium oxide. In this compound, the support may be any support based on aluminium oxide, and more particularly any support where the aluminium oxide is in particular accessible at the surface of said support. The support may accordingly be selected from among supports of relatively homogeneous composition based on aluminium oxide, in particular having a composition based on aluminium oxide which is relatively homogeneous throughout the mass of the support, i.e. from the core up to the surface of the support, and also among heterogeneous supports based on aluminium oxide which comprise aluminium oxide essentially at the surface of the supports. In the case of a heterogeneous support, the support may comprise aluminium oxide deposited, supported or grafted on an inorganic solid which may itself be an inorganic solid support, in particular selected from among metals, oxides or sulfides and salts, for example among silica and metal oxides.

The support may have a specific surface area (BET) selected within a range of from 0.1 to 1000 m²/g, preferably from 0.5 to 800 m²/g. Specific surface area (BET) is measured in accordance with standard ISO 9277 (1995).

The support may in particular comprise aluminium oxide, mixed aluminium oxides or modified aluminium oxides, in particular modified by one or more elements from groups 15 to 17 of the periodic table of the elements.

Aluminium oxide (also referred to as ordinary alumina), is generally taken to mean an aluminium oxide containing substantially no other oxide (or containing less than 2% by weight of one or more other oxides, present in the form of impurities). If it contains more than 2% by weight of one or more other oxides, it is generally agreed to consider the oxide to be a mixed aluminium oxide, i.e. an aluminium oxide combined with at least one other oxide.

The support may preferably comprise aluminium oxide selected from among porous aluminas, non-porous aluminas and mesoporous aluminas.

Porous aluminas are frequently known as “activated aluminas” or alternatively “transition aluminas”. They generally correspond to various partially hydroxylated aluminium oxides, Al₂O₃. These are porous supports generally obtained by an “activation” treatment in particular comprising heat treatment (or dehydration) of a precursor selected from among aluminium hydroxides, such as aluminium tri-hydroxides, hydroxides of aluminium oxide or gel-form aluminium hydroxides. The activation treatment makes it possible to eliminate the water present in the precursor, together with a proportion of the hydroxyl groups, so leaving behind some residual hydroxyl groups and a specific porous structure. The surface of porous aluminas generally comprises a complex mixture of aluminium and oxygen atoms and hydroxyl ions which combine in accordance with specific crystalline forms and which in particular produce both acidic and basic sites. A solid support may be selected from among porous aluminas Y-alumina (gamma-alumina), η-alumina (eta-alumina), δ-alumina (delta-alumina), θ-alumina (theta alumina), K-alumina (kappa-alumina), ρ-alumina (rho-alumina) and X-alumina (chi-alumina), and preferably from among Y-alumina and η-alumina. These various crystalline forms depend essentially on the selection of the precursor and the conditions of the activation treatment, in particular temperature and pressure. The activation treatment may be performed, for example, under a stream of air or a stream of another gas, in particular an inert gas, at a temperature which may be selected within a range of from 100 to 1000° C., preferably from 200 to 1000° C.

It is also possible to use porous or alternatively semi-porous aluminas, produced by an activation treatment as previously described, in particular at a temperature of from 600 to 1000° C. These porous or semi-porous aluminas may comprise mixtures of porous aluminas in at least one of the previously described crystalline forms, such as Y-alumina, ηalumina, δalumina, θ-alumina, K-alumina, ρ-alumina or X-alumina, with a non-porous alumina, in particular α-alumina, in particular in a proportion of 20 to 80% by weight.

Porous aluminas are generally thermal decomposition products of aluminium tri-hydroxides, aluminium oxide hydroxides (or aluminium oxide hydrates) and gel-form aluminium hydroxides (or alumina gels).

Aluminium tri-hydroxides of the general formula Al(OH)₃=Al₂O₃, 3H₂O may exist in various crystalline forms, such as gibbsite or hydrargillite (α-Al(OH)₃), bayerite (β-Al(OH)₃), or nordstrandite. Aluminium tri-hydroxides may be obtained by precipitation from aluminium salts in generally alkaline solutions.

Aluminium oxide hydroxides of the general formula AlO(OH)=Al₂O₃, H₂O may also exist in various crystalline forms, such as diaspore (β-AlO(OH)) or the boehmite (or α-AlO(OH)). Diaspore may be found in certain types of clay and bauxite, and may be synthesised by heat treatment of gibbsite at approximately 150° C., or by hydrothermal treatment of boehmite at 380° C. under a pressure of 50 MPa. Boehmite may readily be obtained by heating the resultant gel-form precipitate with cold treatment of the aluminium salt solutions with ammonia. Aluminium oxide hydroxides may also be obtained by hydrolysis of aluminium alcoholates.

Gel-form aluminium hydroxides (or alumina gels) are generally aluminium polyhydroxides, in particular of the general formula:
nAl(OH)₃, (n−1)H₂O (1)
in which n is a number ranging from 1 to 8. Gel-form aluminium hydroxides may be obtained by one of the methods selected from among thermal decomposition of an aluminium salt, such as aluminium chloride, electrolysis of aluminium salts, such as a mixture of aluminium sulfate and alkali metal sulfate, hydrolysis of aluminium alcoholates, such as aluminium methylate, precipitation from aluminates, such as alkali metal or alkaline-earth metal aluminates, and precipitation from aluminium salts, for example by contacting aqueous solutions of Al₂(SO₄)₃ and ammonia, or of NaAlO₂ and an acid, or of NaAlO₂ and Al₂(SO₄)₃, after which the resultant precipitate may undergo ageing and drying to remove water. Gel-form aluminium hydroxides generally assume the form of an amorphous alumina gel, in particular the form of a pseudoboehmite.

The porous aluminas may have a specific surface area (BET) selected in a range of from 100 to 1000 m²/g, preferably from 300 to 1000 m²/g, in particular from 300 to 800 m²/g, in particular from 300 to 600 m²/g. They may furthermore have a specific pore volume of less than or equal to 1 cm³/g, preferably of less or equal to 0.9 cm³/g, in particular, of less than or-equal to 0.6 cm³/g.

The support may also comprise non-porous aluminas, preferably α-alumina (alpha-alumina), which is generally known as “calcined alumina” or “flame alumina”. α-Alumina exists in the natural state, being known as “corundum”. It may in general be synthesised by heat treatment or calcination of a precursor selected in particular from among aluminium salts, aluminium oxide hydroxides, aluminium tri-hydroxides and aluminium oxides, such as Y-alumina, at a temperature of greater than 1000° C., preferably of greater than 1100° C. It may contain impurities, such as other oxides, for example Fe₂O₃, SiO₂, TiO₂, CaO, Na₂O, K₂O, MgO, SrO, BaO and Li₂O, in proportions of less than 2%, preferably of less than 1% by weight. Non-porous aluminas, such as α-alumina, may have a specific surface area (BET) selected in a range of from 0.1 to less than 300 m²/g, preferably from 0.5 to 300 m²/g, in particular from 0.5 to 250 m²/g.

The support may also comprise mesoporous aluminas, having in particular a specific surface area (BET) selected in a range of from 100 to 800 m²/g. Mesoporous aluminas generally have pores of a width of from 2 nm to 0.05 μm.

The support may also comprise mixed aluminium oxides. Mixed aluminium oxides are generally taken to mean aluminium oxides combined with at least one other oxide in a proportion by weight preferably from 2 to less than 80%, in particular from 2 to less than 50%, in particular from 2 to less than 40% or even from 2 to less than 30%. The other oxide(s) may be oxides of the elements, M, selected from among metals of groups 1 to 13 and elements of group 14, with the exception of carbon, of the periodic table of the elements. More particularly, they may be oxides of the elements M selected from among alkali metals, alkaline-earth metals, transition metals and elements of groups 13 and 14 of said tables, with the exception of carbon. Transition metals generally comprise the metals of groups 3 to 11 of said table, in particular elements 21 to 29, 39 to 47, 57 to 79 (including lanthanides) and actinides. The other oxide(s) of the elements M are preferably selected from among transition metals of groups 3 to 7, lanthanides, actinides and elements of groups 13 and 14 of said table, with the exception of carbon. More particularly, they may be selected from among the oxides of silicon, boron, gallium, germanium, titanium, zirconium, cerium, vanadium, niobium, tantalum, chromium, molybdenum and tungsten.

Mixed aluminium oxides may be selected from among anhydrous aluminates, spinels and aluminosilicates. In particular, anhydrous aluminates may be selected from among anhydrous alkali metal aluminates, such as anhydrous lithium aluminate (LiAlO₂) or anhydrous sodium aluminate (Na₂O, Al₂O₃), and anhydrous alkaline-earth metal aluminates, such as anhydrous tricalcium aluminate (3CaO, Al₂O₃) or anhydrous beryllium aluminate (BeO, Al₂O₃). Spinels may in particular be selected from among aluminium oxides combined with oxides of divalent metals, and in particular from among magnesium spinel (MgAl₂O₄), calcium spinel (CaAl₂O₄, zinc spinel (ZnAl₂O₄, manganese spinel (MnAl₂O₄), iron spinel (FeAl₂O₄) and cobalt spinel (CoAl₂O₄) Aluminosilicates may in particular be selected from among clays, talcum, mica, feldspar, microporous aluminosilicates, in particular molecular sieves, and zeolites.

The support may also comprise modified aluminium oxides, in particular modified by one or more elements from groups 15 to 17, preferably from groups 16 to 17 of the periodic table of the elements, for example phosphorus, sulfur, fluorine or chlorine. The support may in particular comprise alumina superacids or sulfated, sulfided, chlorinated or fluorinated aluminium oxides.

The support may be a support of homogeneous composition, in particular throughout the entire mass of the support. It may also be a heterogeneous support based on aluminium oxide, in which support the aluminium oxide, mixed aluminium oxides or modified aluminium oxides, as previously described, are essentially arranged at the surface of the support, and the core of the support is essentially constituted by an inorganic solid selected in particular from among metals, oxides or sulfides, and salts, such as silica or metal oxides. The heterogeneous support may be prepared by dispersion, by precipitation and/or by grafting of one of the precursors of the above-mentioned compounds based on aluminium oxide onto said inorganic solid. The precursors may in particular be selected from among aluminium hydroxides, in particular from among aluminium tri-hydroxides, aluminium oxide hydroxides and gel-form aluminium hydroxides. Gel-form aluminium hydroxides, as described previously, which are known as alumina gels or amorphous aluminas, are preferred. A heterogeneous support may in particular be produced by processing such a precursor by a sol-gel method or with the assistance of an organometallic compound which in particular facilitates grafting onto the inorganic solid.

The compound according to the invention generally assumes the form of particles which may be of any shape and size, in particular an average size of from 10 nm to 5 mm, preferably from 20 nm to 4 mm. The particles of the support may assume their natural shape or may be shaped so as to have a specific shape, in particular a spherical, spheroidal, hemispherical, hemispheroidal, cylindrical or cubic shape, or may assume the form of rings, tablets, discs or pellets.

The compound according to the invention essentially comprises a tungsten hydride grafted [onto] the support based on aluminium oxide. The oxidation state of the tungsten in the supported metallic compound may have a value selected in a range from 2 to 6, preferably from 4 to 6. The tungsten atom is attached in particular to the solid support, in particular by at least one single bond. It may furthermore be attached to one or more atoms of hydrogen by single bonds (W—H) and optionally to one or more hydrocarbon residues, R, in particular by single or multiple carbon-tungsten bonds. The number of hydrogen atoms attached to an atom of tungsten depends on the oxidation state of tungsten, the number of single bonds attaching said tungsten atom to the support and optionally on the number of single or multiple bonds attaching said tungsten atom to the hydrocarbon residue, R. Thus, the number of hydrogen atoms attached to a tungsten atom may be at least equal to 1 and at most equal to 5, and may preferably range from 1 to 4, preferably from 1 to 3. Grafting of the tungsten hydride onto the solid support based on aluminium oxide is generally taken to mean that the tungsten atom is attached by at least one single bond to said support, and more particularly by at least one single bond (W—OAl) to at least one oxygen atom of the aluminium oxide. The number of single bonds attaching the tungsten atom to the support, in particular by a single bond (W—OAl), depends on the oxidation state of the tungsten and on the number of other bonds attaching the tungsten atom, and is generally equal to 1, 2 or 3.

The tungsten atom of the compound according to the invention may optionally be attached to one or more hydrocarbon residues, R, by one or more single, double or triple carbon-tungsten bonds. The hydrocarbon residues, R, may be identical or different, saturated or unsaturated hydrocarbon residues, in particular comprising from 1 to 20, preferably from 1 to 10 carbon atoms, and optionally comprising silicon, in particular in an organosilane group. They may in particular be selected from among alkyl residues, in particular linear or branched, aliphatic or alicyclic residues, for example alkyl, alkylidene or alkylidyne residues, in particular from C₁ to C₁₀, from among aryl residues, in particular from C₆ to C₁₂, and from among aralkyl, aralkylidene or aralkylidyne residues, in particular from C₇ to C₁₄.

The tungsten atom of the grafted tungsten hydride may be attached to the hydrocarbon residue, R, by one or more single, double or triple carbon-tungsten bonds. The bond may be a single carbon-tungsten bond, in particular of type σ: in this case, the hydrocarbon residue, R, may be an alkyl residue, in particular linear or branched, or an aryl residue, for example the phenyl residue, or an aralkylene residue, for example the benzyl residue or the residue of the formula (C₆H₅—CH₂—CH₂—). An alkyl residue is generally taken to mean a monovalent aliphatic residue originating from the removal of a hydrogen atom from a carbon atom in the molecule of an alkane, or an alkene, or an alkyne, or even of an organosilane, for example, a methyl (CH₃—), ethyl (C₂H₅—), propyl (C₂H₅—CH₂—), neopentyl ((CH₃)₃C—CH₂—), allyl (CH₂═CH—CH₂—), alkynyl (R—C≡C—), in particular ethynyl (CH≡C—), or neosilyl (CH₃)₃Si—CH₂—) residue. The alkyl residue may be, for example, of the formula (R′—CH₂—) where R′ represents a linear or branched alkyl residue.

The bond may also comprise a double carbon-tungsten bond, in particular of type n: in this case, the hydrocarbon residue, R, may be an alkylidene residue, in particular linear or branched, or an aralkylidene residue. An alkylidene residue is generally taken to mean a divalent aliphatic residue originating from the removal of two hydrogen atoms from the same carbon atom in the molecule of an alkane, or an alkene, or an alkyne, or even of an organosilane, for example a methylidene (CH₂═), ethylidene (CH₃—CH═), propylidene (C₂H₅—CH═), neopentylidene ((CH₃)₃C—CH═), or allylidene (CH₂═CH—CH═) residue. The alkylidene residue may be, for example, of the formula (R′—CH═) where R′ represents a linear or branched alkyl residue. An aralkylidene residue is generally taken to mean a divalent aliphatic residue originating from the removal of two hydrogen atoms from the same carbon in an alkyl, alkenyl or alkynyl residue branched from an aromatic group.

The bond may also comprise a triple carbon-tungsten bond: in this case, the hydrocarbon residue, R, may be an alkylidyne residue, in particular linear or branched, or an aralkylidyne residue. An alkylidyne residue is generally taken to mean a trivalent aliphatic residue originating from the removal of three hydrogen atoms from the same carbon atom in the molecule of alkane, or an alkene, or an alkyne, or even of an organosilane, for example an ethylidyne (CH₃—C≡), propylidyne (C₂H₅—C≡), neopentylidyne (CH₃)₃C—C═) or allylidyne (CH₂═CH—C≡) residue. The alkylidyne residue may be, for example, of the formula (R′—C≡), where R′ represents a linear or branched alkyl residue. An aralkylidyne residue is generally taken to mean a trivalent aliphatic residue originating from the removal of three atoms of hydrogen from the same carbon of an alkyl, alkenyl or alkynyl residue branched from an aromatic group.

More particularly, the hydrocarbon residue, R, may be selected from among methyl, ethyl, n-propyl, isopropyl, n-butyl, isobutyl, neopentyl, allyl, neopentylidene, allylidene, neopentylidyne and neosilyl residues.

The tungsten atom of the compound according to the invention may be complexed by one or more hydrocarbon ligands, in particular aromatic or carbonyl ligands.

The tungsten hydride grafted onto the support based on aluminium oxide may be represented schematically by the following formula: 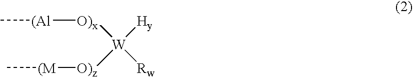
in which W, Al, O and H respectively represent atoms of tungsten, aluminium, oxygen and hydrogen, M represents an atom of one or more elements of another oxide, as defined previously, R represents a hydrocarbon residue, as defined previously, and x, y, w and z are integers, the sum of which (w+x+y+z) equals 2 to 6, and with x=1 to 3, y =1 to 5, w=0 to 4 and z=0 to 2. In the formula (2), the −(Al—O) and -(M-O) bonds represent one or more single or multiple bonds respectively attaching the aluminium atom and the atom M to one of the atomic constituents of the support based on aluminium oxide, in particular to one of the oxygen atoms of this support.

Under infrared spectroscopy, the compound according to the invention generally exhibits one or more absorption bands which are specific to the (W—H) bond, the frequency of which bands may vary depending on the coordination sphere of the tungsten and in particular may depend on the number of bonds of the tungsten with the support, with the hydrocarbon residues R and with other hydrogen atoms. Accordingly, at least two absorption bands have been found at 1903 and 1804 cm⁻¹, these bands in particular being specific to the (W—H) bond under consideration, in particular in the environment of the (W—OAl) bonds attaching the same tungsten atom to an oxygen atom which is itself attached to an aluminium atom of an α-alumina. By way of comparison, tungsten hydride grafted under the same conditions onto a silica support generally exhibits under infrared spectroscopy at least one of the two absorption bands at 1940 and 1960 cm⁻¹, these bands differing from the previous ones and in particular being specific to the (W—H) bond under consideration, in particular in the environment of the (W—OSi) bonds attaching the same tungsten atom to an oxygen atom which is itself attached to a silicon atom of the silica support.

Another method capable of characterising the presence of a (W—H) bond in the compound according to the invention is provided by a proton nuclear magnetic resonance measurement (solid ¹H-NMR) at 500 MHz, where the value of the tungsten hydride chemical shift (δ_{W-H}) is equal to 10.6 ppm (parts per million).

The compound according to the invention may furthermore comprise an aluminium hydride, in particular at the surface of the support and in particular in the vicinity of the grafted tungsten hydride. It is thought that an aluminium hydride may be formed by opening of an aluminoxane bridge (of the formula Al—O—Al) which is in particular present at the surface of the support and by reaction between a hydrogen atom of a grafted tungsten hydride and the aluminoxane bridge opened in this manner. A simple test for characterising the aluminium hydride present in the compound of the invention beside a tungsten hydride comprises a deuteration reaction of said compound. The test may be performed by contacting the compound according to the invention with a deuterium atmosphere under an absolute pressure of 66.7 kPa, at a temperature selected between 25 and 80° C., preferably equal to 60° C., for a period of 15 minutes. A selective deuteration reaction is thus, performed under these conditions, the reaction enabling the replacement of the hydrogen atoms in the (W—H) bond with deuterium atoms, so forming new (W-D) bonds which, under infrared spectroscopy, have two absorption bands at 1293 and 1393 cm⁻¹, while leaving the hydrogen atoms in the (Al—H) bonds unchanged, these bonds then being characterised under infrared spectroscopy by an absorption band at 1914 cm⁻¹.

The present invention also relates to a method for production of the supported metallic compound. The compound according to the invention, which essentially assumes the form of a tungsten hydride grafted onto a support based on aluminium oxide, may be prepared by a method comprising the following steps:

(1) a dispersion and grafting step of an organometallic tungsten precursor (Pr) onto a support based on aluminium oxide, in which precursor the tungsten is in particular attached or complexed to at least one hydrocarbon ligand, so as to form a hydrocarbon compound or complex of tungsten grafted onto said support, then

(2) a hydrogenolysis step of the grafted hydrocarbon compound or complex of tungsten, arising from the previous step, so as to form a tungsten hydride grafted onto said support.

The organometallic tungsten precursor, Pr, preferably comprises a tungsten atom attached or complexed to one or more hydrocarbon ligands. The tungsten atom may in particular be attached to a carbon of the hydrocarbon ligand by single, double or triple (carbon-tungsten) bonds. The hydrocarbon ligands may be identical or different, saturated or unsaturated hydrocarbon residues, in particular aliphatic or alicyclic residues, preferably from C₁ to C₂₀, in particular from C₁ to C₁₀, and may be selected in particular from among the above-described hydrocarbon residues, R. The number of hydrocarbon ligands attached to the tungsten atom depends on the oxidation state of tungsten in the precursor Pr and may be at most equal to the oxidation state of the tungsten in the precursor Pr, in particular may be greater than 0 and at most equal to the maximum oxidation state of tungsten and may preferably have any value of from 2 to 6, in particular from 4 to 6.

The precursor Pr may comprise a tungsten atom which is in particular complexed to one or more hydrocarbon ligands, the oxidation state of the tungsten being in this case equal to zero. The hydrocarbon ligand may be selected from among aromatic ligands or carbonyl ligands. The precursor Pr may accordingly be selected from among bis-arene tungsten and hexacarbonyl tungsten.

Prior to the first dispersion and grafting step, the support based on aluminium oxide may be subjected to a prior calcination and/or dehydroxylation step. Calcination of the support may be performed in such a manner as to oxidise the carbon optionally present in the support and to eliminate it in the form of carbon dioxide. It may be performed by subjecting the support to an oxidising heat treatment, in particular under a stream of dry air, at a temperature below the sintering temperature of the support, for example at a temperature of from 100 to 1000° C., preferably of 200 to 800° C., for a duration sufficient to eliminate the carbon dioxide which may range from 0.1 to 48 hours, under a pressure of less than, equal to or greater than atmospheric pressure.

The support may also be subjected to another prior step, known as dehydroxylation. This step may be performed in such a manner as optionally to eliminate the residual water from the support and a proportion of the hydroxyl groups, to leave behind, in particular at the surface of the support, a residual quantity of hydroxyl groups and optionally to form aluminoxane bridges (of the formula Al—O—Al). Dehydroxylation may be performed by subjecting the support to heat treatment under a stream of inert gas, for example under a stream of nitrogen, argon or helium, under a pressure which is preferably below atmospheric pressure, for example under an absolute pressure of from 10⁻⁴ Pa to 10² kPa, preferably from 10⁻² Pa to 50 kPa, at a temperature below the sintering temperature of the support, for example at a temperature of from 100 to 1000° C., preferably from 200 to 800° C., and for a duration sufficient to leave behind an appropriate residual quantity of hydroxyl groups and/or aluminoxane in the support which may range from 0.1 to 48 hours. The dehydroxylation step may advantageously be performed after the calcination step.

The dispersion and grafting step may be performed by sublimation, by impregnation with the assistance of a solvent or by dry mixing. In the case of a sublimation step, the precursor Pr, which generally assumes the solid state under normal conditions, is heated in particular under a pressure of below atmospheric pressure and under temperature conditions ensuring its sublimation and migration in the gaseous state onto the support. Sublimation may be performed at a temperature of from −30 to 200° C., and in particular under an absolute pressure of from 10⁻⁴ to 1 Pa. Grafting of the precursor Pr onto the support may be monitored by infrared spectroscopy. Any excess precursor Pr which has not grafted onto the support may be removed by inverse sublimation.

The dispersion and grafting step may also be performed by impregnation with the assistance of a solvent. In this case, the precursor Pr may be dissolved in a polar or non-polar organic solvent, for example pentane or ethyl ether. Impregnation may be performed by contacting the support based on aluminium oxide with the previously prepared solution of the precursor Pr. Impregnation may be performed at a temperature of from −80 to 200° C., under an inert atmosphere, for example an atmosphere of nitrogen, argon or helium, and preferably with stirring. In this manner, a suspension of a hydrocarbon compound or complex of tungsten grafted onto the support is obtained. Any excess precursor Pr which has not grafted onto the support may be removed by washing with an organic solvent, which may be identical to or different from that used during impregnation.

The dispersion and grafting step may also be performed by dry mixing, in particular mechanical dry mixing, in the absence of liquid or liquid solvent. In this case, the precursor Pr which assumes the form of a solid, is mixed with the support based on aluminium oxide, in the absence of liquid or liquid solvent, in particular with mechanical stirring and under an inert atmosphere, for example an atmosphere of nitrogen, argon or helium, so as to form a mixture of two solids. During or after dry mixing, heat treatment and/or treatment under a pressure below atmospheric pressure may be performed so as to cause the precursor Pr to migrate and react with the support. Any precursor which has not been grafted onto the support may be removed by inverse sublimation or by washing with organic solvent.

Production of the compound according to the invention may comprise a second step known as hydrogenolysis. This comprises a hydrogenolysis reaction of the hydrocarbon compound or complex of tungsten grafted onto the support, as prepared in the preceding step. The reaction is generally performed so as to form a tungsten hydride grafted onto the support. Hydrogenolysis is generally taken to mean a cleavage reaction of a molecule with attachment of hydrogen onto the two cleaved portions. Specifically, the cleavage reaction in particular occurs between the tungsten atom grafted onto the support and the carbon atom of the precursor Pr fixed or complexed with said tungsten atom. Hydrogenolysis may be performed with the assistance of hydrogen or a reducing agent, capable in particular of converting the grafted hydrocarbon compound or complex of tungsten into grafted tungsten hydride. Hydrogenolysis may be performed by contacting the grafted hydrocarbon compound or complex of tungsten with the hydrogen or reducing agent. It may be performed under an atmosphere of hydrogen or an inert atmosphere when a reducing agent is used, under an absolute pressure of from 10⁻² to 10 MPa, at a temperature of from 20 to 500° C., for a period of from 0.1 to 48 hours.

The present invention furthermore relates to the use of the compound according to the invention in a method making use of hydrocarbon cleavage and recombination reactions. It relates more particularly to the use of the compound according to the invention in a method for manufacturing hydrocarbon(s) having a modified carbon skeleton by reaction of at least one aliphatic hydrocarbon with itself, or with at least one other aliphatic hydrocarbon, or alternatively with at least one aromatic or cyclanic hydrocarbon substituted by at least one alkyl residue. In this method, an aliphatic hydrocarbon may be used which is selected from among linear aliphatic hydrocarbons, in particular from C₂ to C₃₀, preferably from C₂ to C₂₀, and branched aliphatic hydrocarbons, in particular from C₄ to C₃₀, preferably from C₄ to C₂₀, or an aromatic hydrocarbon substituted by at least one alkyl residue selected from among substituted aromatic hydrocarbons from C₇ to C₃₀, preferably from C₇ to C₂₀, with at least one linear or branched alkyl residue, in particular from C₁ to C₂₄, preferably from C₁ to C₁₄, or a cyclanic hydrocarbon substituted by at least one alkyl residue selected from among substituted cyclanic hydrocarbons from C₄ to C₃₀, preferably from C₄ to C₂₀, with at least one linear or branched alkyl residue, in particular from C₁ to C₂₇, preferably from C₁ to C₁₇. Such a method is in particular described in international patent application WO 98/02244. The method may be performed at a temperature of from 20 to 600° C., preferably from 50 to 500° C., under an absolute pressure of from 0.1 to 100 MPa, preferably from 0.1 to 50 MPa. It may be performed preferably in the presence of hydrogen or of an agent which forms hydrogen “in situ”, for example under a hydrogen partial pressure of from 0.01 to 50 MPa, preferably from 0.1 to 20 MPa. The compound according to the invention in particular acts as a catalyst, in particular as a catalyst for the hydrocarbon metathesis reaction. It may be reactivated or regenerated by being contacted with hydrogen or any agent which forms hydrogen “in situ”, during or separately from the hydrocarbon manufacturing method.

The compound according to the invention may in particular be used as a hydrocarbon, in particular alkane, metathesis reaction catalyst. Particularly remarkably, it exhibits extremely high catalytic activity in hydrocarbon metathesis and/or homologation (or disproportionation) reactions, and very high selectivity for the formation of normal hydrocarbons (i.e. with a linear chain) in comparison with the formation of branched-chain hydrocarbons, in particular “iso” form hydrocarbons. The compound according to the invention in particular exhibits particularly high catalytic activity in the alkane metathesis and/or homologation (or disproportionation) reactions and simultaneously high selectivity for n-alkanes in comparison with the iso-alkanes which are formed.

The compound according to the invention may in particular also be used as a catalyst in a method for manufacturing hydrocarbon(s) by reaction of methane with at least one other aliphatic hydrocarbon, or with at least one aromatic or cyclanic hydrocarbon substituted by at least one alkyl residue. Such a method is described in international patent application WO 01/04077. The method in particular comprises contacting methane with at least one of the above-stated hydrocarbons in the presence of the supported metallic compound according to the invention. The reactions arising from such contacting are generally hydrocarbon metathesis reactions comprising hydrocarbon cleavage and recombination reactions and simultaneously reactions incorporating methane into these hydrocarbons. These reactions are generally known by the term “methane-olysis” reaction. In this method, methane may be used with at least one other aliphatic hydrocarbon which is selected from among linear aliphatic hydrocarbons, in particular from C₂ to C₃₀, preferably from C₃ to C₂₀, and branched aliphatic hydrocarbons, in particular from C₄ to C₃₀, preferably from C₄ to C₂₀, or an aromatic hydrocarbon substituted by at least one alkyl residue selected from among substituted aromatic hydrocarbons from C₇ to C₃₀, preferably from C₇ to C₂₀, with at least one linear or branched alkyl residue, in particular from C₁ to C₂₄, preferably from C₁ to C₁₄, or a cyclanic hydrocarbon substituted by at least one alkyl residue selected from among substituted cyclanic hydrocarbons from C₄ to C₃₀, preferably from C₄ to C₂₀, with at least one linear or branched alkyl residue, in particular from C₁ to C₂₇, preferably from C₁ to C₁₇. In this method, a mixture of methane with one or more other aliphatic and/or cyclanic hydrocarbons may also be used, such as natural gas, liquefied petroleum gas or LPG, wet gas or wet natural gas (i.e. a mixture of methane with C₂ to C₅ or C₃ and/or C₄ alkanes), natural gas liquids or NGL, or light hydrocarbons fractions from C₁ to C₆, or from C₁ to C₅, or from C₁ to C₄, or from C₁ to C₃, or from C₁ to C₂. The method may be performed at a temperature of from 20 to 600° C., preferably of 50 to 500° C., in particular under a partial methane pressure of from 0.1 to 100 MPa, preferably from 0.1 to 50 MPa, and optionally in the presence of hydrogen or an agent which forms hydrogen “in situ”, for example under a hydrogen partial pressure of from 0.01 to 50 MPa, preferably of 0.1 to 20 MPa.

The compound according to the invention may in particular also be used as a catalyst in a method for manufacturing alkane(s), in particular ethane, by reacting methane with itself. This amounts more specifically to a method comprising contacting methane with the compound according to invention. This method is generally known as methane to ethane conversion. In this case, the methane conversion method is in particular a non-oxidising conversion method, in particular performed by catalytic coupling of methane, enabling methane to be converted essentially into ethane, in particular with extremely high selectivity for ethane. The method may be performed at a temperature of from 20 to 800° C., preferably from 50 to 600° C., under an absolute pressure of from 0.01 to 100 MPa, preferably from 0.1 to 50 MPa.

The compound according to the invention may also be used in a method for manufacturing hydrocarbon(s) by a crossed metathesis reaction between at least one starting hydrocarbon and said compound. Such a method is in particular described in international patent application WO 00/27781. The crossed metathesis reaction is the reaction which is in particular obtained by cleaving the hydrocarbon residue or ligand attached or complexed to the tungsten hydride of the compound according to invention and by recombining said residue or ligand with at least one other residue originating from cleavage of the starting hydrocarbon. In this method, a starting hydrocarbon may be used which is selected from among aliphatic linear or branched hydrocarbons in particular from C₂ to C₃₀, preferably from C₂ to C₂₀, and cyclanic hydrocarbons substituted by at least one alkyl residue, in particular from C₄ to C₃₀, preferably from C₄ to C₂₀, the alkyl residue being linear or branched, in particular from C₁ to C₂₇, preferably from C₁ to C₁₇. The compound according to the invention in particular comprises at least one hydrocarbon residue R or a hydrocarbon ligand attached to the tungsten hydride. The method may be performed at a temperature of from 20 to 500° C., preferably from 50 to 400° C., under an absolute pressure of from 0.01 to 50 MPa, preferably from 0.1 to 20 MPa.

The compound according to the invention may in particular also be used as a catalyst in method for manufacturing hydrocarbon(s) or hydrocarbon oligomer(s) or polymer(s) with a modified carbon skeleton by reaction of a starting hydrocarbon polymer with hydrogen. Such a method is in particular described in European patent application EP 0 840 771. The starting hydrocarbon polymer may be a (co)polymer of one or more olefinic or vinyl monomers, in particular a polyolefin such as a polyethylene, a polypropylene, a polybut-1-ene, a polyisobutene, a copolymer of ethylene with at least one C₃ to C₈ alpha-olefin, a copolymer of propylene with at least one C₄ to C₈ alpha-olefin, a copolymer of isobutene with but-1-ene, or an aromatic polyvinyl such as a polystyrene or poly(alpha-methylstyrene). The hydrocarbon polymer may have a weight average molecular weight, Mw, of from 10³ to 10⁷, preferably from 10⁴ to 10⁶. The method may be performed by contacting the starting polymer with the supported metallic compound according to the invention, in the presence of hydrogen and optionally a solvent medium in particular capable of solubilising the starting polymer, or under temperature conditions which allow the starting polymer to be in the molten state during contacting. The method may be performed at a temperature of from 20 to 400° C., preferably from 50 to 300° C., under a hydrogen partial pressure of from 0.001 to 20 MPa, preferably from 0.01 to 10 MPa, for a period ranging in particular from 5 minutes to 100 hours, preferably from 10 minutes to 50 hours. The method may in particular be performed in a field of increasing centrifugal force, for example from 5 to 1000 times greater than the earth's force of gravity, in particular in a revolving disc reactor. In this case, the duration of the method may be from 1 second to 5 minutes, preferably from 2 seconds to 2 minutes.

Use of the compound according to the invention is particularly advantageous in one of the above-described methods, since a considerable increase in catalytic activity of this compound is observed in carbon-carbon, carbon-hydrogen and optionally tungsten-carbon bond cleavage and recombination reactions, in particular in. hydrocarbon, in particular alkane, metathesis reactions. Furthermore, in alkane metathesis reactions, the compound according to the invention exhibits extremely high selectivity for n-alkanes in comparison with the iso-alkanes which are formed.

The following Examples illustrate the present invention.

### EXAMPLE 1

### Production of a Tungsten Hydride Grafted onto an Alumina

In a prior step, 530 mg of an α-alumina having an average size of 40 μm and a specific surface area (BET) of 200 m²/g, containing 90% by weight of alumina and 9% by weight of water, and sold by Johnson Matthey (Great Britain), are subjected to calcination treatment under a stream of dry air at 500° C. for 15 hours, then to dehydroxylation treatment under an absolute pressure of 10⁻² Pa at 500° C. for 15 hours, such that the alumina calcined and hydroxylated in said manner exhibits, under infrared spectroscopy, three absorption bands respectively at 3774, 3727 and 3683 cm⁻¹ which are in particular characteristic of the residual (AlO—H) bond.

In a first step, the 530 mg of previously prepared alumina are introduced into a glass reactor under an argon atmosphere and at 25° C., followed by a solution of 6 ml of n-pentane containing 300 mg of tungsten tris(neopentyl)neopentylidyne used as a precursor Pr and of the general formula:
W[—CH₂—C(CH₃)₃]₃[≡C—C(CH₃)₃] (3)

The resultant mixture is maintained at 25° C. for 3 hours. At the end of this time, an organometallic tungsten compound grafted onto alumina is obtained, the excess precursor Pr which has not reacted being removed by washing with n-pentane at 25° C. The organometallic tungsten compound grafted in this manner is dried under a vacuum. It contains 1.5% by weight of tungsten and is of the general formula:
(Al—O)ₓW[—CH₂—C(CH₃)₃]_{y}[═CH—C(CH₃)] (4)

with x=1 and y=2.

In a second step, 40 mg of the previously obtained grafted organometallic tungsten compound are isolated and subjected in a glass reactor to hydrogenolysis treatment by contacting with hydrogen under an absolute hydrogen pressure of 73 kPa at 150° C. for 15 hours. At the end of this time, the reactor is cooled to 25° C., a compound (W/Al-1) according to the invention which in particular comprises a tungsten hydride grafted onto alumina is obtained and isolated under argon. The compound (W/Al-1) contains 1.5% by weight of tungsten and, under infrared spectroscopy, exhibits two absorption bands respectively at 1903 and 1804 cm⁻¹, which are characteristic of the (W—H) bond in particular grafted onto alumina.

### EXAMPLE 2

### Production of a Tungsten Hydride Grafted onto an Alumina

The prior calcination and dehydroxylation steps of the α-alumina are exactly identical to those in Example 1.

In a first step, 53 mg of the previously prepared alumina are isolated and introduced into a glass reactor at 25° C. under one atmosphere of argon. The precursor Pr of the general formula (3) as used in Example 1 is then introduced into the reactor. The reactor is then heated to 70° C. for 2 hours, so as to sublime the precursor Pr onto the alumina and to form an organometallic tungsten compound grafted onto alumina. At the end of this time, the excess precursor Pr which has not reacted is removed by inverse sublimation at 70° C. The reactor is then cooled to 25° C. and an organometallic tungsten compound grafted in this manner which contains 3.7% by weight of tungsten and is of the general formula (4) above is isolated under argon.

The second step is performed in exactly the same manner as in Example 1, except that-the organometallic tungsten compound grafted onto alumina prepared in the preceding step is used. In this manner, a compound (W/Al-2) according to the invention is obtained comprising a tungsten hydride grafted onto alumina and containing 3.7% by weight of tungsten. Under infrared spectroscopy, it exhibits two absorption bands respectively at 1903 and 1804 cm⁻¹ which are characteristic of the (W—H) bond in particular grafted onto alumina.

Compound (W/Al-2) is subjected to a selective deuteration test demonstrating that it comprises a tungsten hydride and an aluminium hydride, both grafted onto the alumina. A sample of compound (W/Al-2) is placed in a glass reactor, is then contacted in this reactor with a deuterium atmosphere under an absolute pressure of 66.7 kPa at a temperature of 60° C. for 15 minutes. At the end of this time, the reactor is cooled to 25° C. and the solid compound deuterated in this manner is isolated under argon, said compound exhibiting under infrared spectroscopy an absorption band at 1914 cm⁻¹, which is characteristic of the (Al—H) bond which has not been changed by the deuteration reaction performed under these conditions. It is furthermore observed that the absorption bands at 1903 and 1804 cm⁻¹, which are characteristic of the (W—H) bond grafted onto the alumina, disappear in favour of the absorption bands respectively at 1293 and 1393 cm⁻¹, which are characteristic of the (W-D) bond grafted onto alumina and formed by the deuteration reaction of the (W—H) bonds.

### EXAMPLE 3

### Production of a Tungsten Hydride Grafted onto an Alumina

The prior calcination and dehydroxylation steps of the α-alumina are exactly identical to those in Example 1.

In a first step, 2 g of the previously prepared alumina are isolated and introduced under an argon atmosphere into a glass reactor at 25° C. equipped with a magnetic stirrer bar. 305 mg of the precursor Pr of the general formula (3) as used in Example 1 are then introduced into this reactor. The reactor is heated to 66° C. and the resultant dry-prepared mixture is stirred for 4 hours. At the end of this time, the reactor is cooled to 25° C., then the mixture of solid is washed with n-pentane at 25° C. The solid compound washed in this manner is dried in a vacuum, then isolated under argon so as to obtain an organometallic tungsten compound grafted onto alumina containing 3.9% by weight of tungsten and of the above general formula (4).

The second step is performed exactly as in Example 1, except that the previously prepared organometallic tungsten compound grafted onto alumina is used. In this manner, a compound (W/Al-3) according to the invention is obtained comprising a tungsten hydride grafted onto alumina and containing 3.9% by weight of tungsten. Under infrared spectroscopy, it exhibits two absorption bands respectively at 1903 and 1804 cm⁻¹ which are characteristic of the (W—H) bond grafted onto alumina. Furthermore, when examined by nuclear magnetic resonance (solid ¹H-NMR) at 500 MHz, it exhibits a tungsten hydride chemical shift value (δ_{W-H}) equal to 10.6 ppm (parts per million).

### EXAMPLE 4 (COMPARATIVE)

### Production of a Tungsten Hydride Grafted onto a Silica

In a prior step, 44 mg of a silica sold under the trade name “Aerosil 200”® by Degussa (Germany), having a BET specific surface area of 200 m²/g, are subjected to dehydroxylation treatment under an absolute pressure of 10⁻² Pa, at 700° C. for 15 hours, such that the silica dehydroxylated in this manner exhibits under infrared spectroscopy an absorption band at 3747 cm⁻¹, which is in particular characteristic of the residual (SiO—H) bond.

In a first step, 44 mg of the previously prepared silica are isolated and introduced into a glass reactor at 25° C. under an argon atmosphere. The precursor Pr of the general formula (3) as used in Example 1 is then introduced into the reactor. The reactor is then heated to 70° C. for 2 hours, so as to sublime the precursor Pr onto the silica and to form an organometallic tungsten compound grafted onto silica. At the end of this time, the excess precursor Pr which has not reacted is removed by inverse sublimation at 70° C. The reactor is then cooled to 25° C. and an organometallic tungsten compound grafted in this manner onto silica which contains 5.5% by weight of tungsten and is of the following general formula is isolated under argon:
(Si—O)ₓW[—CH₂—C(CH₃)₃]y[—C—C(CH₃)] (5)

with x=1 and y=2.

In a second step, the organometallic tungsten compound grafted onto the silica prepared in the preceding step is subjected to hydrogenolysis treatment by contacting with hydrogen under a pressure of 73 kPa at 150° C. for 15 hours. At the end of this time, a compound (W/Si-1) comprising a tungsten hydride grafted onto silica and containing 5.5% by weight tungsten is obtained by way of comparison and isolated under argon; under infrared spectroscopy, it exhibits an absorption band at 1940 cm⁻¹ which is characteristic of the (W—H) bond in particular grafted onto silica.

### EXAMPLE 5 (COMPARATIVE)

### Production of a Tantalum Hydride Grafted onto an Alumina.

Exactly the same procedure is followed as in Example 2, except that in the first step 50 mg of the alumina prepared during the prior steps are isolated and that, instead of precursor Pr, tantalum tris(neopentyl)neopentylidene is introduced into the reactor as precursor Pr′, of the general formula:
Ta[—CH₂—C(CH₃)₃]₃[=CH—C(CH₃)₃] (6)

An organometallic tantalum compound grafted onto alumina containing 5.6% by weight of tantalum is obtained in this manner.

The second step is performed in exactly the same manner as in Example 2, except that the previously prepared organometallic tantalum compound grafted onto alumina is used. In this manner, a compound (Ta/Al-1) comprising a tantalum hydride grafted onto alumina and containing 5.6% by weight of tantalum is obtained by way of comparison. Under infrared spectroscopy, it exhibits an absorption band at 1830 cm⁻¹, which is characteristic of the (Ta—H) bond grafted onto alumina, together with another band at 1914 cm.⁻¹, which is in particular characteristic of the (Al—H) bond.

### EXAMPLE 6 (COMPARATIVE)

### Production of a Tantalum Hydride Grafted onto a Silica.

Exactly the same procedure is followed as in Example 4, except that in the first step 50 mg of the alumina prepared during the prior step are isolated and that, instead of precursor Pr, tantalum tris(neopentyl)neopentylidene is introduced into the reactor as precursor Pr′, of the general formula (6). An organometallic tantalum compound grafted onto silica containing 5.5% by weight of tantalum is obtained in this manner.

The second step is performed in exactly the same manner as in Example 4, except that the previously prepared organometallic tantalum compound grafted onto silica is used. In this manner, a compound (Ta/Si-1) comprising a tantalum hydride grafted onto silica and containing 5.5% by weight of tantalum is obtained by way of comparison. Under infrared spectroscopy, it exhibits an absorption band at 1830 cm⁻¹, which is characteristic of the (Ta—H) bond grafted onto silica.

### EXAMPLE 7

### Propane Metathesis

The supported metallic compounds (W/Al-3), (W/Si-1), (Ta/Al-1) and (Ta/Si-1) prepared respectively in Examples 3, 4, 5 and 6 are successively used in a propane metathesis reaction which may be represented by following equation:
2 C₃H₈→C₂H₆+C₄H₁₀ (7)

Each propane metathesis reaction is performed under the following conditions. The supported metallic compound is prepared “in situ” in a glass reactor as described above. The reactor is then evacuated, after which it is filled with propane up to a pressure of 76 kPa and is heated to 150° C. A mixture essentially comprising ethane and n and iso-butanes, and also a smaller quantity of methane, of n and iso-pentane, and even some C₆ homologues in a very small quantity is then observed to form.

For each of the tests performed with the supported metallic compounds, the cumulative number (CN) of moles of propane converted over time per mole of tungsten or tantalum of the supported metallic compound, is measured and calculated, this being performed at the end of 120 hours of reaction.

Furthermore, for each of these tests, the ratio of selectivity (SnC₄) of the n-butane formation reaction to the selectivity (SiC₄) of the iso-butane formation reaction is measured and calculated at the end of 120 hours. The selectivities (SnC₄) and (SiC₄) are respectively calculated according to the following equations:
SnC4=(number of moles of n-butane formed)/(total number of moles of alkanes formed) and (8)
SiC4=(number of moles of iso-butane formed)/(total number of moles of alkanes formed) (9)

Tables 1 summarises the results of the above-mentioned measurements and calculations for each of the propane metathesis tests performed.

TABLE 1

| | Supported metallic | | |
|---|---|---|---|
| Tests | compound | CN | SnC₄/SiC₄ |
| 1 | W/Al-3 | 180 | 10 |
| 2 | W/Si-1 | 10 | 12 |
| (comparative) | | | |
| 3 | Ta/Al-1 | 39 | 7 |
| (comparative) | | | |
| 3 | Ta/Si-1 | 60 | 2.7 |
| (comparative) | | | |

Analysis of the results in Table 1 shows that the supported metallic compound according to the invention (W/Al-3) exhibits extremely high catalytic activity in the propane metathesis reaction which is very much greater than the catalytic activities of the other compounds, with a selectivity ratio between n-butane and iso-butane formed which corresponds to a high level.

### EXAMPLE 8

### “Methane-olysis” of Propane

A mixture comprising 800 mol of propane per 10⁶ mol of methane is continuously passed at a flow rate of 1.5 ml/min under a partial methane pressure of 5 MPa through a reactor of a capacity of 5 ml, heated to 250° C. and containing 300 mg of the supported metallic compound according to the invention (W/Al-3) prepared in Example 3,

The propane “methane-olysis” reaction may be written according to the following equation:
CH₄+C₃H₈→2 C₂H₆ (10)

Ethane is indeed observed to form over time.

1. A supported metallic compound comprising a support based on aluminium oxide onto which a tungsten hydride is grafted.

2. A compound according to claim 1, characterised in that the support is selected from among supports with a homogenous composition based on aluminium oxide and from among heterogeneous supports based on aluminium oxide comprising aluminium oxide essentially at the surface of said supports.

3. A compound according to claim 1 or claim 2, characterised in that the support has a specific surface area (BET) selected in a range of from 0.1 to 1000 m²/g, preferably from 0.5 to 800 m²/g.

4. A compound according to any one of claims 1 to 3, characterised in that the support comprises aluminium oxide, mixed aluminium oxides or modified aluminium oxides, in particular modified by one or more elements from groups 15 to 17 of the periodic table of the elements.

5. A compound according to claim 4, characterised in that the support comprises aluminium oxide selected from among porous aluminas, non-porous aluminas and mesoporous aluminas.

6. A compound according to claim 5, characterised in that the porous alumina is selected from among Y-alumina, ηalumina, δ-alumina, θ-alumina, K-alumina, ρ-alumina and X-alumina, preferably from among Y-alumina and η-alumina.

7. A compound according to claim 6, characterised in that the porous alumina has a specific surface area (BET) in a range of from 100 to 1000 m²/g, preferably from 300 to 1000 m²/g, in particular from 300 to 800 m²/g.

8. A compound according to claim 5, characterised in that the non-porous alumina is α-alumina.

9. A compound according to claim 8, characterised in that the non-porous alumina has a specific surface area (BET) in a range of from 0.1 to 300 m²/g, preferably from 0.5 to 300 m²/g, in particular from 0.5 to 250 m²/g.

10. A compound according to claim 6 or claim 7, characterised in that the porous alumina comprises a mixture of one or more crystalline forms of porous aluminas with α-alumina, in particular in a proportion by weight of from 20 to 80%.

11. A compound according to claim 4, characterised in that the mixed aluminium oxides are selected from among aluminium oxides combined with at least one other oxide in a proportion by weight of preferably from 2 to less than 80%, in particular from 2 to less than 50%, in particular from 2 to less than 40%.

12. A compound according to claim 11, characterised in that the other oxide(s) are oxides of the elements, M, selected from among the metals of groups 1 to 13 and the elements of group 14, with the exception of carbon, of the periodic table of the elements.

13. A compound according to claim 11, characterised in that the other oxide(s) are selected from among oxides of alkali metals, of alkaline-earth metals, of transition metals and of the elements of groups 13 and 14, with the exception of carbon, of the periodic table of the elements.

14. A compound according to claim 4, characterised in that the modified aluminium oxides comprise one or more of the elements of groups 16 or 17 of the periodic table of the elements, and are preferably selected from among superacids of alumina and sulfated, sulfided, fluorinated and chlorinated aluminium oxides.

15. A compound according to any one of claims 1 to 14, characterised in that it assumes the form of particles having an average size of from 10 nm to 5 mm, preferably from 20 nm to 4 mm.

16. A compound according to any one of claims 1 to 15, characterised in that the oxidation state of the tungsten has a value selected in a range of from 2 to 6, preferably from 4 to 6.

17. A compound according to any one of claims 1 to 16, characterised in that the tungsten atom is attached to one or more hydrogen atoms and optionally to one or more hydrocarbon residues, R.

18. A compound according to claim 17, characterised in that the hydrocarbon residues R are identical or different, saturated or unsaturated hydrocarbon residues, comprising in particular from 1 to 20, in particular from 1 to 10 carbon atoms and optionally comprising silicon.

19. A compound according to any one of claims 1 to 18, characterised in that the tungsten atom is complexed by one or more hydrocarbon ligands, in particular aromatic ligands or carbonyl ligands.

20. A compound according to any one of claims 1 to 19, characterised in that, under infrared spectroscopy, it exhibits at least one of the two absorption bands at 1903 and 1804 cm⁻¹.

21. A compound according to any one of claims 1 to 20, characterised in that, when examined by proton nuclear magnetic resonance (solid ¹H-NMR) at 500 MHz, it exhibits a tungsten hydride chemical shift value (δ_{W-H}) equal to 10.6 ppm.

22. A method for production of the compound according to any one of claims 1 to 21, characterised in that it comprises (1) a dispersion and grafting step of an organometallic tungsten precursor, Pr, onto a support based on aluminium oxide, in which precursor the tungsten is in particular attached or complexed to at least one hydrocarbon ligand, so as to form a hydrocarbon compound or complex of tungsten grafted onto said support, then (2) a hydrogenolysis step of the grafted hydrocarbon compound or complex of tungsten, arising from the preceding step, so as to form a tungsten hydride grafted onto said support.

23. A method according to claim 22, characterised in that the support based on aluminium oxide is subjected to a prior calcination and/or dehydroxylation step.

24. A method according to claim 22 or claim 23, characterised in that the dispersion and grafting step is performed by sublimation, by impregnation with the assistance of a solvent, or by dry mixing.

25. A method according to any one of claims 22 to 24, characterised in that the hydrogenolysis step is performed by contacting the grafted hydrocarbon compound or complex of tungsten with hydrogen or a reducing agent.

26. Use of the compound according to any one of claims 1 to 21 in a method making use of hydrocarbon cleavage and recombination reactions.

27. Use of the compound according to any one of claims 1 to 21 as a hydrocarbon, in particular alkane, metathesis reaction catalyst.

28. Use of the compound according to any one of claims 1 to 21 in a method for manufacturing hydrocarbon(s) having a modified carbon skeleton by the reaction of at least one aliphatic hydrocarbon with itself, or with at least one other aliphatic hydrocarbon, or with at least one aromatic or cyclanic hydrocarbon substituted by at least one alkyl residue.

29. Use according to claim 28, characterised in that the aliphatic hydrocarbon is selected from among linear aliphatic hydrocarbons, in particular from C₂ to C₃₀, and branched aliphatic hydrocarbons, in particular from C₄ to C₃₀, the aromatic hydrocarbon substituted by at least one alkyl residue is selected from among substituted aromatic hydrocarbons from C₇ to C₃₀ with at least one linear or branched alkyl residue, in particular from C₁ to C₂₄, and the cyclanic hydrocarbon substituted by at least one alkyl residue is selected from among substituted cyclanic hydrocarbons from C₄ to C₃₀ with at least one linear or branched alkyl residue, in particular from C₁ to C₂₇.

30. Use of the compound according to any one of claims 1 to 21 in a method for manufacturing hydrocarbon(s) by reaction of methane with at least one other aliphatic hydrocarbon, or with at least one aromatic or cyclanic hydrocarbon substituted by at least one alkyl residue.

31. Use of the compound according to any one of claims 1 to 21 in a method for manufacturing alkane(s), in particular ethane, by reaction of methane with itself.

32. Use of the compound according to any one of claims 1 to 21 in a method for manufacturing hydrocarbon(s) by a crossed metathesis reaction between at least one starting hydrocarbon and said compound.

33. Use of the compound according to any one of claims 1 to 21 in a method for manufacturing hydrocarbon(s) or hydrocarbon oligomer(s) or polymer(s) with a modified carbon skeleton by reaction of a starting hydrocarbon polymer with hydrogen.
